(19) United States
(12) Patent Application Publication
(10) Pub. No.: US 2020/0061261 A1
(43) Pub. Date: Feb. 27, 2020

(54) Device For The Initial Treatment of Wounds

(71) Applicant: Petar Ilic, Klagenfurt (AT)

(72) Inventor: Petar Ilic, Klagenfurt (AT)

(21) Appl. No.: 16/329,542

(22) PCT Filed: Aug. 31, 2017
(86) PCT No.: PCT/AT2017/000060

§ 371 Date: Nov. 15, 2019

(30) Foreign Application Priority Data
Aug. 31, 2016 (AT) A 401/2016

(51) Int. Cl. A61M 1/04; A61M 1/00

(52) U.S. Cl. CPC A61M 1/04; A61M 1/0027; A61M 1/0031; A61M 1/0088

(57) ABSTRACT

A device for the initial treatment of wounds, which has two chambers each open on one side. The first chamber is designed as a vacuum chamber having a valve for sucking off air and peripherally surrounds the substantially centrally arranged second chamber which is intended to be placed over the wound. In a preferred embodiment of the device according to the present teaching, an opening provided with a valve for sucking off air or body fluids is provided on the second chamber.

### TECHNICAL FIELD

The present teaching relates to a device for the initial treatment of wounds, in particular for the stanching of bleedings and for the treatment of life-threatening injuries, such as chest injuries for the treatment of tension pneumothorax.

### BACKGROUND

An effective and rapid treatment of serious and life-threatening bleedings in the event of injuries from physical attacks, traffic accidents and other types of accidents, but also in the case of war, terrorist attacks or natural disasters, is very important. For example, in the case of injury to blood vessels, there exist many initial treatment means, such as stanching of limbs, application of a pressure bandage, constriction of blood vessels, application of chemical granules or gels to stop bleeding. The most commonly used method is probably the compression bandage, which has been proven for some time. But even with the use and application of a pressure bandage one can quickly reach his/her limits and there may be other disadvantages, for example, if a first aider alone is dealing with the initial treatment of wounds. For example, it is difficult or impossible for a first aider alone to apply a pressure bandage on a lying person having a thigh injury. It would therefore be desirable to have a device for the initial treatment of wounds, which is constructed so that it can easily be applied by only one person, even if one is the injured person himself/herself.

If there is heavy bleeding in an unfavorable part of the body, such as in the pelvic area or in the shoulder area, applying a pressure bandage is also difficult to achieve and often not effective enough, since usually not enough pressure can be exerted against a wound to stop bleeding from it. For such cases, it would be desirable to have a device for the initial treatment of wounds that adheres to the skin without pressure being applied against the wound.

In the event of an injury comprising a foreign object in the wound, any pressure applied to the injured area may be detrimental. There is a risk of pushing a foreign object even deeper into an injury, which can make it worse. In addition, this also brings much more pain for the injured with it. In the event that a foreign object protrudes a little out of an injury, the first aider has the option of extracting it or applying pressure against the injury to stop the bleeding. Both methods can be a major disadvantage. In such events, a device would be desirable, with which such a bleeding can be stopped without necessarily having to remove the foreign object or to press it even deeper into the injury.

Having to deal with many injured people at the same time, such as after a terrorist attack, a military operation or a natural disaster, the time required to provide first aid to all the injured people is a major challenge. The above methods can take a long time, which is very disadvantageous when only a few first aiders are on site, who must provide first aid to many injured people. It would therefore be desirable to have a device whose application takes only a short period of time, for example a few seconds.

Furthermore, a device for the initial treatment of wounds is desirable, the construction and use of which is simple, so that even non-trained and non-qualified first aiders can provide a safe and effective initial treatment of wounds and thus do not make mistakes which are dangerous for an injured person.

In the initial treatment of, for example, injuries to people who are trapped in vehicles, buried under objects or buried, often only a very limited space is available, while for wound care in conventional manner a larger space is required. For example, it is very difficult, if not impossible, to treat a bleeding thigh injury of a person trapped inside a vehicle. Therefore, it is desirable to have a device that can provide treatment of a bleeding injury even when it is difficult to reach or can be reached only with one hand or there is no space to put pressure on the bleeding wound.

Another problem with the treatment of wounds in general is the required sterility. The applied products, such as a compression bandage, are sterile packed, but after being applied to an injury, they do not maintain it 100% sterile. It is therefore desirable to have a device for the initial treatment of wounds which can not only be sterilized and packaged in a sterile manner, but also shields an injury completely from the outside after its application, so that there is little or no risk of infection when transporting an injured person. This device is also intended to prevent foreign substances from entering an injury, regardless of whether you have to transport an injured person through water, a sandstorm or for an injured person particularly painful, salt water. In addition, it would be desirable to have a device for the initial treatment of wounds by which a bleeding wound may be also treated underwater.

Another particular problem is thorax injuries. For thorax injuries which entail a tension pneumothorax, a patch or occlusive dressing can be used, as it is known for example under the name “Chest Seal”. Via a valve attached to it, air in the pleural space can be removed by the patient's breathing. For an improved device for the initial treatment of wounds of thorax injuries, it is also desirable not only to provide such a valve through which air, blood or other substances can be removed from a pleural space by the injured person's breathing, but to provide such a valve, which can be connected also with a vacuum pump. This makes it possible to pump substances and material out of a pleural space if an injured person does not manage to drain them by breathing. It is also possible to assist an injured person with such a process by pumping out. Furthermore, it is important with such injuries that the device can be used without major pressure against the injury, as pressure against an injured thorax can cause further dangers to the injured person.

### SUMMARY

The present teaching aims at providing a device of the type as at the beginning, which overcomes the above mentioned disadvantages.

This aim is achieved by a device for the initial treatment of wounds, which has two chambers each open on one side, wherein a first chamber is designed as a vacuum chamber having a valve for sucking off air and peripherally surrounds a substantially centrally arranged second chamber which is intended to be placed over the wound.

Preferably, an opening provided with a valve for sucking off air or body fluids is provided on the second chamber on the device for the initial treatment of wounds.

In order to further improve the adhesion of device for the initial treatment of wounds and the closure of the wound to the environment, the first chamber and the second chamber may have sealing lips on the areas of the body which is in contact with a body surface.

For monitoring and adjusting the pressure, a vacuum gauge may preferably be provided in the device at the first chamber.

The advantages of the device according to the present teaching will become apparent from the following observations and the description of the drawings.

The device according to the present teaching is applied very quickly when used, it takes far less time than for example for the application of a pressure bandage. This is very important for only one injured person, but also if several injured people are to be cared for at the same time.

The device according to the present teaching is safe, in the use of which virtually no serious mistakes can be made. An emergency doctor is not always available for first aid in the event of life-threatening injuries, and first aiders often make the mistake of applying a pressure bandage too firm for the initial treatment of wounds, which causes a risk of congestion and too much pain for the patient.

The device is very easy to use, it is placed over a wound and by means of a vacuum pump the air is pumped out from the first chamber.

The device can be used by the first aider alone. An initial treatment by the injured person himself/herself is also possible. Due to the resulting vacuum, the device is fixed to the injury, the injured person does not necessarily have to be lifted or rotated, which often requires two or more first aiders. For the purposes of the present teaching herein and throughout the description a vacuum is to be understood as a pressure of less than the atmospheric pressure.

By means of the device according to the present teaching, bleedings to the outside are stopped in such a way that the blood flow is not interrupted in the event of injuries of the blood vessels. This can be a decisive advantage for certain injuries. Especially with injuries of blood vessels in the neck area interrupting the blood flow is very dangerous. If the blood flow to the brain is interrupted or reduced, this can lead to brain damage or brain death after a short time. In addition, pressure applied to the neck area may interfere with breathing.

The fact that when using the device according to the present teaching, no direct pressure is exerted on a wound, proves to be very advantageous even if foreign objects are found in a wound. Applying pressure to and around a wound would in such events push a foreign object deeper into the tissue and possibly worsen the wound. For a patient, this also brings much higher pain.

The small required space when applying the device according to the present teaching is another decisive advantage, for example, with injured people trapped in vehicles or partially buried. Here a first aider must be able to reach an injury only with one arm, since the device can be connected with vacuum pumps also over a longer connection tube. Thus, the vessel can be pressed with one hand to the wound and at the same time with the other hand, the air can be pumped out.

With the device according to the present teaching there is also the possibility to treat injuries under water.

After applying the device according to the present teaching, the wound is enclosed by a chamber. If the device has been sterilized and has been sterile packed in a conventional manner and the wound is sterilized prior to application, the wound is then enclosed within a sterile area. In addition, no other substances or materials can come into contact with the wound from the outside, such as water, sand, dust, germs and bacteria.

In the case of thorax injuries, it proves to be a decisive advantage that the device according to the present teaching adheres to the body surface/skin through a resulting vacuum and does not have to be glued to the thorax, such as, for example, a dressing in the form of a so-called “Chest Seal”. Substances such as water, blood, sweat, etc., which impair an adhesive effect, cause the reverse effect when using the device according to the present teaching, they further improve the adhesion to the body. In the event of injuries to thorax, it is particularly advantageous when using the device according to the present teaching that no additional pressure against a wound has to be applied to the injured thorax, as it is the case, for example, when applying a “Chest Seal”.

For thorax injuries, the further embodiment of the device according to the present teaching with a valve on the second chamber, which can also be connected to a vacuum pump, is particularly advantageous. This makes it possible to pump out substances such as air, water, blood or wound secretion via the wound from the pleural space after the device according to the present teaching has been applied to a thorax injury which has resulted in pulmonary collapse.

The use and opening of the valve of the second chamber thus also makes it possible to treat thorax injuries that result in pulmonary collapse. This is an important advantage since with this preferred embodiment of the device according to the present teaching several types of injuries may be treated.

### BRIEF DESCRIPTION OF THE DRAWINGS

The device according to the present teaching will be further described below with reference to the accompanying drawings.

FIG. 1 shows a cross section through an embodiment of the device according to the present teaching.

FIG. 2 shows a bottom view of the device shown in FIG. 1.

FIG. 3 shows a cross section through a further embodiment of the device according to the present teaching, which additionally has sealing lips.

FIG. 4 shows a bottom view of the device shown in FIG. 3.

FIG. 5 shows a cross section through a further embodiment of the device according to the present teaching as shown in FIGS. 3 and 4, which additionally comprises a vacuum gauge.

### DETAILED DESCRIPTION

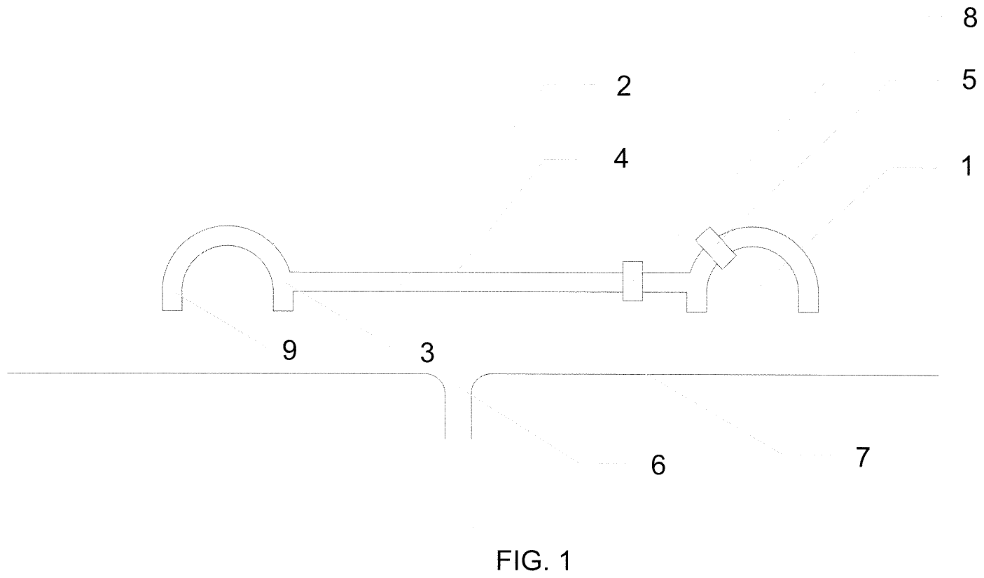

Referring to FIG. 1, an embodiment of the device according to the present teaching comprises two chambers 1, 2 each open on one side, which are separated by a side wall 3 from each other. The first chamber 1 is designed as a vacuum chamber and surrounds the second chamber 2 peripherally. The second chamber 2 thus constitutes an inner chamber 2 and has a ceiling 4.

The first chamber 1 has a valve 10, via which by means of a vacuum pump (not shown) air can be withdrawn. This creates a vacuum in the chamber 1. When used for the treatment of wounds, the device according to the present teaching is placed above a bleeding wound 6 on the body surface 7 before the air is pumped off, and in such a way that the second chamber 2 surrounds the wound 6. The wound 6 is then enclosed by the second chamber 2, then the first chamber 1 is deprived of air by means of the vacuum pump. Thus, the device adheres on the body surface/skin 7 and prevents further leaking of blood from the chamber 2. On the ceiling 4 of the second chamber 2, a valve 8 is attached, which is closed and can be opened. The valve 8 is opened only to treat a tension pneumothorax after an open injury to the thorax, so that after opening the valve 8, for example air or blood obstructing respiration may be removed via the valve 8. A vacuum pump (not shown) can also be connected to the valve 8, so that, if necessary, for example air or blood can be pumped out of the wound 6. The chamber 1 has in the form shown in FIG. 1 side walls 9 and 3, which are formed so that the first chamber has a semi-circular cross-section. The shape of the side walls 9 and 3 and the cross sections of the chamber 1 are not limited thereby. Rather, they can vary depending on the field of application or depending on the material used to make the device according to the present teaching. An essential feature of the device according to the present teaching is that the second chamber 2 is peripherally surrounded by the first chamber 1.

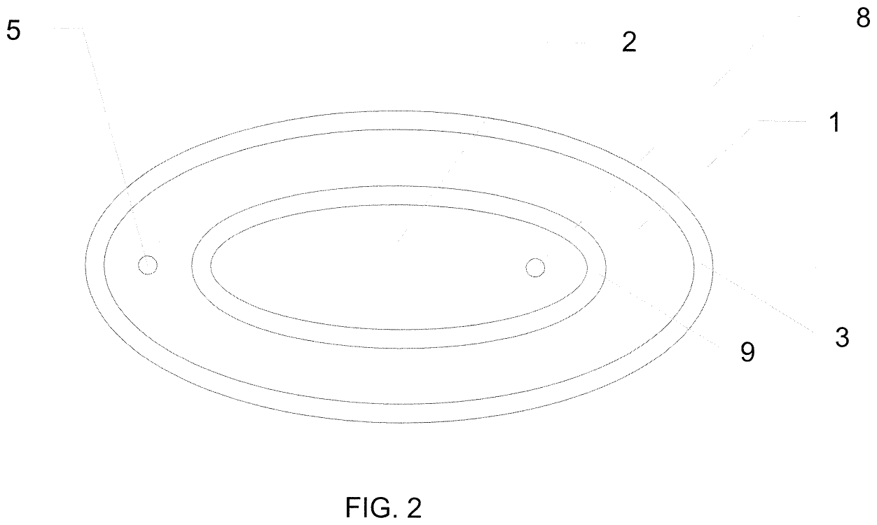

As shown in FIG. 2, the device according to the present teaching has an oval base in the illustrated embodiment. This base is one of many possible bases and is not limiting. Rather, the base can vary and, for example, be circular or polygonal. In particular, the base of the device according to the present teaching can be made variable depending on the size and location of a possible injury. Likewise, the size of the device according to the present teaching and the size ratio of the chambers 1 and 2 to each other can vary.

The embodiment shown in FIGS. 1 and 2 can also be modified in that no valve is provided on the second chamber 2. In this case, the device according to the present teaching can be used as intended and advantageously for the initial treatment of wounds, but is only partially suitable for special use with thorax injuries, since no leaking or sucking off, for example, air, blood, wound secretion or other substances can take place.

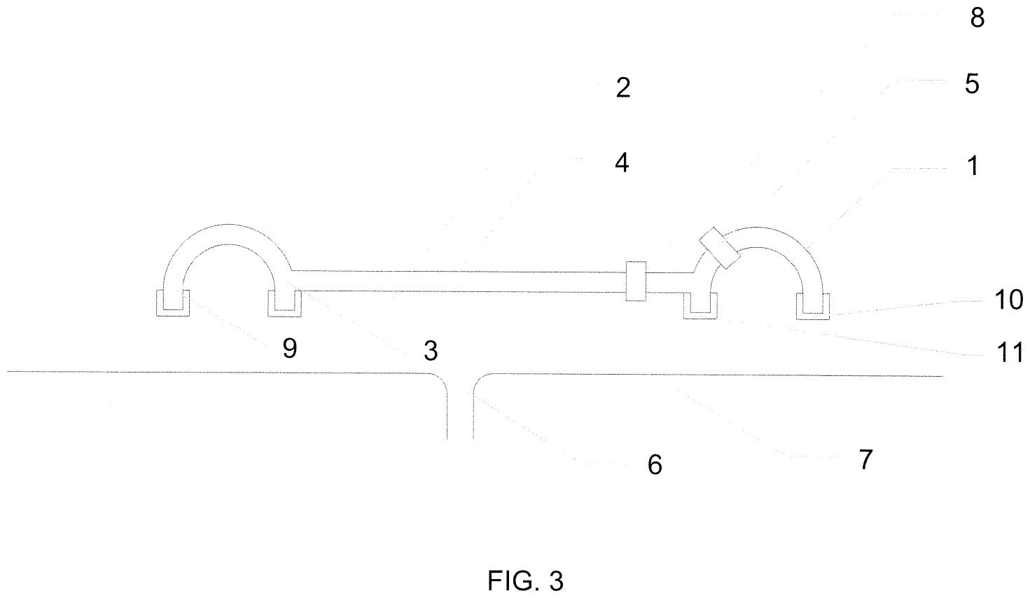

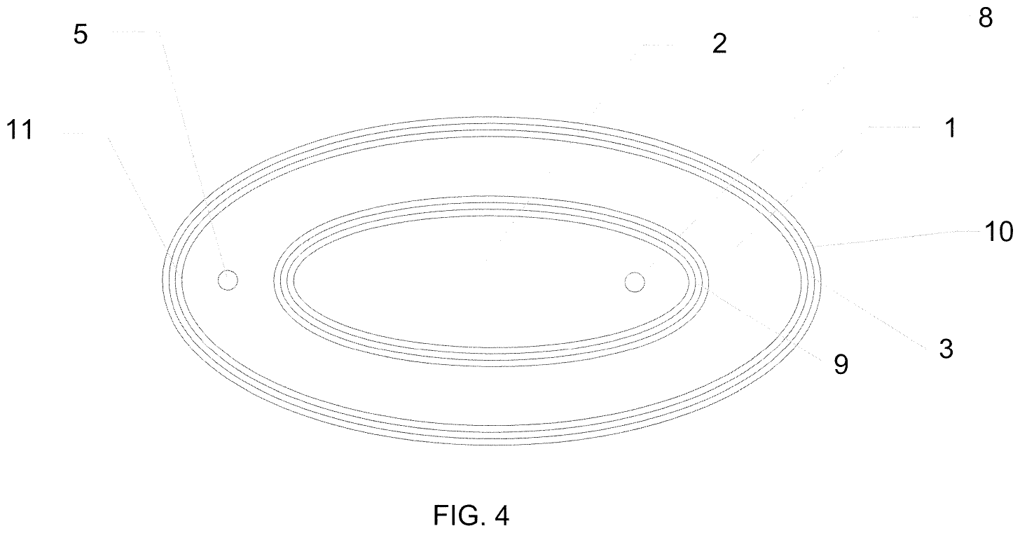

In the embodiment shown in FIG. 3, sealing lips 10, 11 are provided on the open side of the chambers 1, 2, which further improve the sealing of the device according to the present teaching against penetration of substances and materials from the outside and the adhesion to the body surface. The sealing lips 10 and 11 are attached to the underside of the side walls 9 and 3. The base of this embodiment in FIG. 4 is that of an oval.

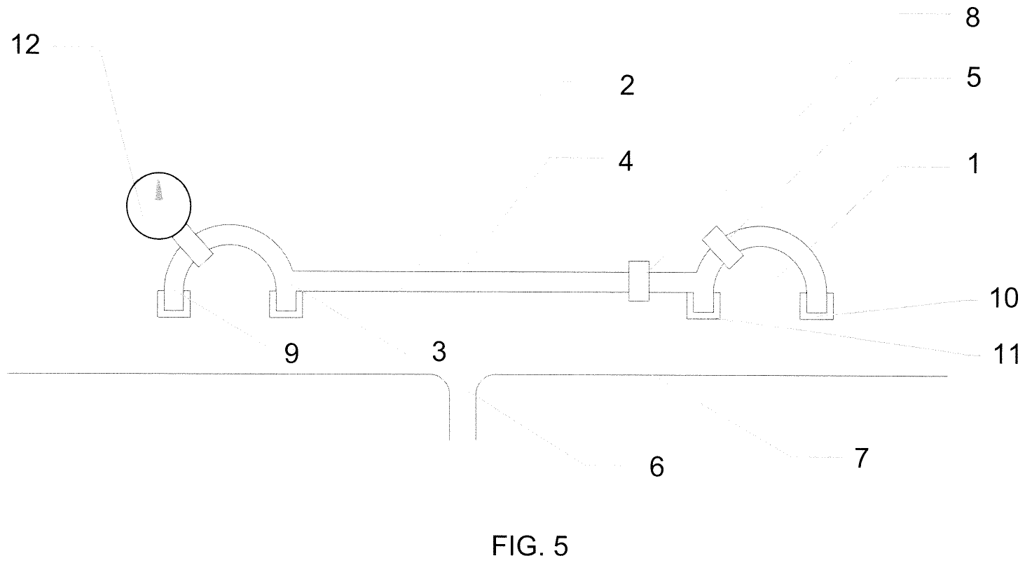

FIG. 5 shows a further form of the embodiment shown in FIGS. 3 and 4. On the chamber 1, a valve 5 is provided, which can be connected to a vacuum pump. On the ceiling 4 of the chamber 2, a valve 8 is also provided. In the opened state, this is a check valve to the outside, which can also be connected to a vacuum pump. At the valve 8, a pressure pump (not shown) can also be attached. The first chamber 1 has a vacuum gauge 12 which provides information about the strength and permanence of a vacuum resulting when using the device according to the present teaching. On the side walls 9 and 3 sealing lips 10 and 11 are provided.

The device according to the present teaching can be made of any suitable material which is suitable for medical use. It is preferably a plastically or elastically deformable plastic. If necessary, the device according to the present teaching can also be equipped with metal wires or metal straps. For example, the use of metal clips in the first chamber 1 could prevent the chamber 1 from collapsing as the vacuum increases.

When used, the device according to the present teaching is placed over an outwardly open injury 6 in such a way that the second chamber 2 completely surrounds the injury. If necessary, the device according to the present teaching is easily pressed against the body surface. If now the vessel is in contact with the skin 7 of the body surface, the air can be withdrawn via the valve 5 of the first chamber 1 by means of a vacuum pump. The vacuum produced in the first chamber 1 presses the device according to the present teaching firmly against the skin 7, whereby the wound 6 is enclosed in the second chamber 2 and a continued loss of blood from the second chamber 2 is avoided.

If the injury is an outwardly open thorax injury resulting in a tension pneumothorax (pulmonary collapse), the valve 8 provided in the second chamber 2 is opened, the air present in the pleural space escapes from the second chamber 2 into the environment. Penetration of air into the pleural space via the second chamber 2 is no longer possible, so that the lung can decompress by breathing.

If decompression by the injured person's breathing is not possible, the valve 8 can be connected to a vacuum pump to suck off air, blood, wound secretion or other substances.

The vacuum gauge 12 serves to control the strength and permanency of a vacuum produced in the first chamber 1 when the device according to the present teaching is used.

1. A device for the initial treatment of wounds, wherein it has two chambers each open on one side, wherein the first chamber is designed as a vacuum chamber having a valve for sucking off air and peripherally surrounds the substantially centrally arranged second chamber which is intended to be placed over the wound.

2. The device for the initial treatment of wounds according to claim 1, wherein an opening provided with a valve for sucking off air or body fluids is provided on the second chamber.

3. The device for the initial treatment of wounds according to claim 1, wherein the chambers have sealing lips on the areas which are in contact with a body surface.

4. The device for the initial treatment of wounds according to claim 1, wherein a vacuum gauge is provided at the first chamber 1.
